(19) United States
(12) Patent Application Publication
(10) Pub. No.: US 2005/0048649 A1
(43) Pub. Date: Mar. 3, 2005

(54) Method for preparing polypeptides variants

(75) Inventor: Jens Sigurd Okkels, Tokyo (JP)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/738,669

(22) Filed: Nov. 2, 2004

Related U.S. Application Data
(63) Continuation of application No. 10/188,594, filed on Jul. 3, 2002, now abandoned.
Application No. 10/188,594 is a continuation of application No. 09/008,363, filed on Jan. 16, 1998, now abandoned.
Application No. 09/008,363 is a continuation of application No. PCT/DK96/00343, filed on Aug. 12, 1996.

(30) Foreign Application Priority Data
Aug. 11, 1995 (DK) 0907/95
Sep. 20, 1995 (DK) 1047/95

(51) Int. Cl. C12N 15/85; C12N 15/74

(52) U.S. Cl. 435/455; 435/471

(57) ABSTRACT

The present invention relates to a method for preparing positive polypeptide variants by shuffling different nucleotide sequences of homologous DNA sequences by in vivo recombination comprising the steps of (a) forming at least one circular plasmid comprising a DNA sequence encoding a polypeptide, (b) opening said circular plasmid(s) within the DNA sequence(s) encoding the polypeptide(s), (c) preparing at least one DNA fragment comprising a DNA sequence homologous to at least a part of the polypeptide coding region on at least one of the circular plasmid(s), (d) introducing at least one of said opened plasmid(s), together with at least one of said homologous DNA fragment(s) covering full-length DNA sequences encoding said polypeptide(s) or parts thereof, into a recombination host cell, (e) cultivating said recombination host cell, and (f) screening for positive polypeptide variants.

### CROSS-REFERENCE TO RELATED APPLICATIONS

This application is a continuation of U.S. application Ser. No. 10/188,594, filed Jul. 3, 2002, which is a continuation of U.S. application Ser. No. 09/008,363, filed Jan. 16, 1998, which is a continuation of PCT/DK96/00343, filed Aug. 12, 1996, which claims priority under 35 U.S.C. 119 of Danish application nos. 0907/95 and 1047/95, filed Aug. 11, 1995 and Sep. 20, 1995, respectively, the contents of which are fully incorporated herein by reference.

### FIELD OF THE INVENTION

The present invention relates to a method for preparing polypeptide variants by in vivo recombination.

### BACKGROUND OF THE INVENTION

The advantages of producing biologically active polypeptides by cloning naturally occurring DNA sequences from microorganisms, such as fungal organisms and bacteria using recombinant DNA technology have been known for quite some years.

Preparation of novel polypeptide variants and mutants, such as novel modified enzymes with altered characteristics, e.g. specific activity, substrate specificity, pH-optimum, pI, Kₘ, Vₘₐₓ etc., have especially during the recent years diligently and successfully been used for obtaining polypeptides with improved properties.

For instance, within the technical field of enzymes the washing and/or dishwashing performance of e.g. proteases, lipases, amylases and cellulases have been improved significantly.

In most cases these improvements have been obtained by site-directed mutagenesis resulting in substitution, deletion or insertion of specific amino acid residues which have been chosen either on the basis of their type or on the basis of their location in the secondary or tertiary structure of the mature enzyme (see for instance U.S. Pat. No. 4,518,584).

An alternative general approach for modifying proteins and enzymes have been based on random mutagenesis, for instance, as disclosed in U.S. Pat. No. 4,894,331 and WO 93/01285

As it is a cumbersome and time consuming process to obtain polypeptide variants or mutants with improved functional properties a few alternative methods for rapid preparation of modified polypeptides have been suggested.

Weber et al., (1983), Nucleic Acids Research, vol 11, 5661-5661, describes a method for modifying genes by in vivo recombination between to homologous genes. A linear DNA sequence comprising a plasmid vector flanked to a DNA sequence encoding alpha-1 human interferon in the 5′-end and a DNA sequence encoding alpha-2 human interferon in the 3′-end is constructed and transfected into a rec A positive strain of E. coli. Recombinants were identified and isolated using a resistance marker.

Pompon et al., (1989), Gene 83, p. 15-24, describes a method for shuffling gene domains of mammalian cytochrome P-450 by in vivo recombination of partially homologous sequences in Saccharomyces cerevisiae by transforming Saccharomyces cerevisia with a linearized plasmid with filled-in ends, and a DNA fragment being partially homologous to the ends of said plasmid.

Stemmer, (1994), Proc. Natl. Acad. Sci. USA, Vol. 91, 10747-10751; Stemmer, (1994), Nature, vol. 370, 389-391, concern methods for shuffling homologous DNA sequences by an in vitro PCR method. One cycle of shuffling consists of digesting a pool of homologous genes with DNase I. The resulting small fragments are reassembled into full-length genes. Positive recombinant genes containing shuffled DNA sequences are selected from a DNA library based on their improved function. Positive recombinants can be used as the starting material for (an)other shuffling round(s).

U.S. Pat. No. 5,093,257 (Assignee: Genencor Int. Inc.) discloses a method for producing hybrid polypeptides by in vivo recombination. Hybrid DNA sequences are produced by forming a circular vector comprising a replication sequence, a first DNA sequence encoding the amino-terminal portion of the hybrid polypeptide, a second DNA sequence encoding the carboxy-terminal portion of said hybrid polypeptide. The circular vector is transformed into a rec positive microorganism in which the circular vector is amplified. This results in recombination of said circular vector mediated by the naturally occurring recombination mechanism of the rec positive microorganism, which include prokaryotes such as Bacillus and E. coli, and eukaryotes such as Saccharomyces cerevisiae.

Despite the existence of the above methods there are still need for even better iterative in vivo recombination methods for preparing novel positive polypeptide variants.

### SUMMARY OF THE INVENTION

The object of the present invention is to provide an improved method for preparing positive polypeptide variants by an in vivo recombination method.

The inventor of the present invention have surprisingly found that such positive polypeptide variants may advantageously be prepared by shuffling different nucleotide sequences of homologous DNA sequences by in vivo recombination comprising the steps of
a) forming at least one circular plasmid comprising a DNA sequence encoding a polypeptide,
b) opening said circular plasmid(s) within the DNA sequence(s) encoding the polypeptide(s),
c) preparing at least one DNA fragment comprising a DNA sequence homologous to at least a part of the polypeptide coding region on at least one of the circular plasmid(s),
d) introducing at least one of said opened plasmid(s), together with at least one of said homologous DNA fragment(s) covering full-length DNA sequences encoding said polypeptide(s) or parts thereof, into a recombination host cell,
e) cultivating said recombination host cell, and
f) screening for positive polypeptide variants.

### BRIEF DESCRIPTION OF THE DRAWINGS

FIG. 1 shows the yeast expression plasmid pJSO26 comprising DNA sequence encoding the Humicola lanuginosa lipase gene.

FIG. 2 shows the yeast expression plasmid pJSO37, comprising DNA sequence encoding the Humicola lanuginosa lipase gene containing twelve additional restriction sites.

FIG. 3 shows the plasmid pJSO26.

FIG. 4 shows the plasmid pJSO37.

FIG. 5 shows the in vivo recombination of the 0.9 kb synthetic wild-type Humicola lanuginosa lipase with pJSO37 using Saccharomyces cerevisiae as the recombination host cell (described in Example 1).

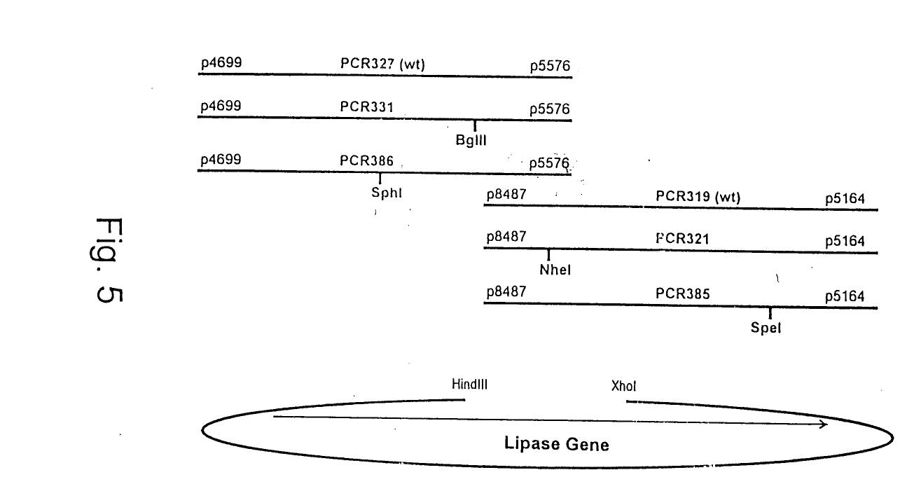

FIG. 6 shows the in vivo recombination of a DNA fragment prepared from Humicola lanuginosa lipase variant (y) with Humicola lanuginosa lipase variant (d) comprised in a plasmid using Saccharomyces cerevisiae as the recombination host cell (described in Example 2).

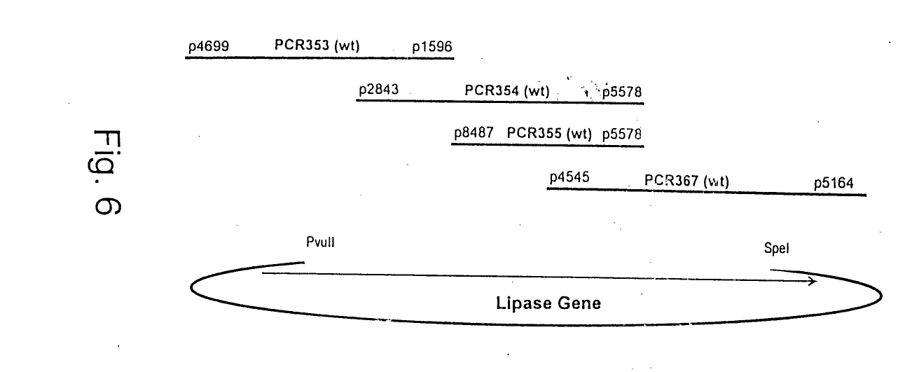

FIG. 7 shows an overview over the location of the inactivation site of the Humicola lanuginosa lipase gene and the number of the clone (referred to as “blue number” in the tables). Location of restriction enzyme sites and clone numbers are relative to the initiation codon of the Lipolase gene. In all cases a stop codon was located in the new reading frame 10 to 50 bp from the frameshift.

FIG. 8 shows an overview of the creation of active Humicola lanuginosa lipase genes from the recombinations in Table 2A and 2B by a “mosaic mechanism”. Lines indicate the introduction of the fragment sequence into the vector and lines with a x indicate sequences that are not introduced in the active lipase colonies. The primers used for the PCR fragment are shown together with the location of the frameshift mutation (marked by the restriction site used for the construction).

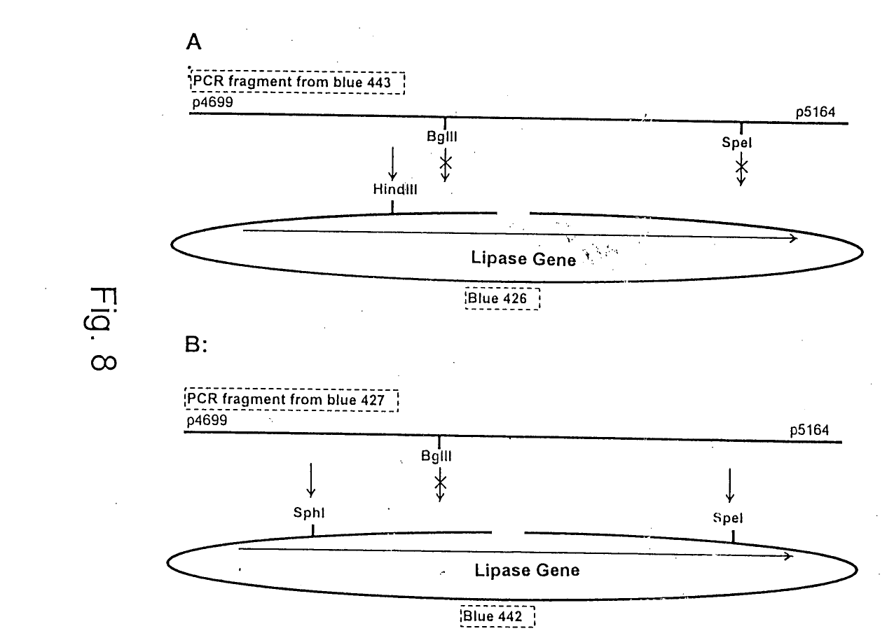

FIG. 9 shows an overview of fragments used in the recombination of 2 partial overlapping fragments into a gapped vector. The primers used for the PCR fragments are shown together with the location of the frameshift mutation (if not wild type).

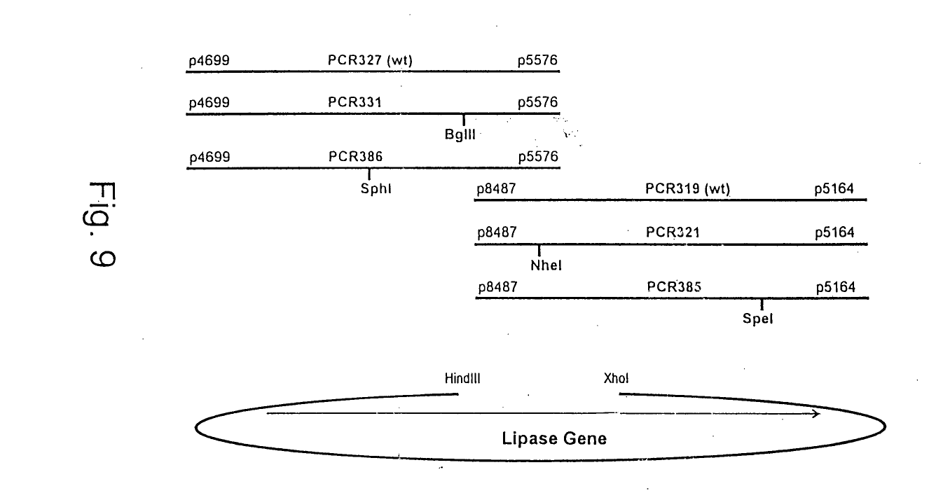

FIG. 10 shows an overview of fragments used in the recombination of 3 partial overlapping fragments into a gapped vector. The primers used for the PCR fragments are shown. The overlap between fragment PCR353 and fragment PCR355 is about 10 bp.

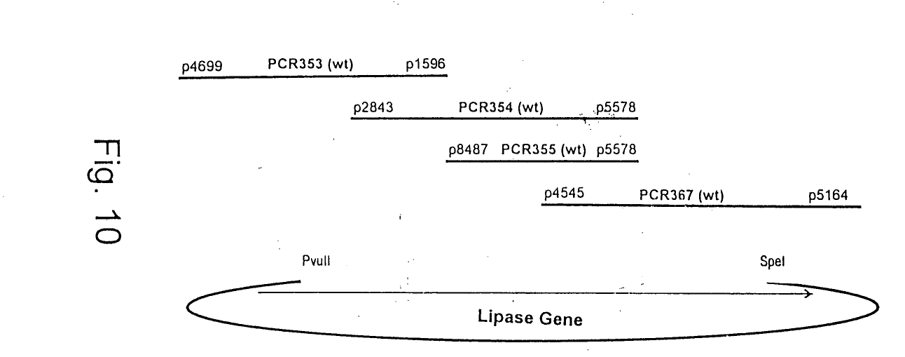

### DETAILED DESCRIPTION OF THE INVENTION

The object of the present invention is to provide an improved method for preparing positive polypeptide variants by an iterative in vivo recombination method.

The inventor of the present invention have surprisingly found an efficient method for shuffling homologous DNA sequences in an in vivo recombination system using a eukaryotic cell as a recombination host cell.

A “recombination host cell” is in the context of the present invention a cell capable of mediating shuffling of a number of homologous DNA sequences.

The term “shuffling” means recombination of nucleotide sequence(s) between two or more homologous DNA sequences resulting in output DNA sequences (Le. DNA sequences having been subjected to a shuffling cycle) having a number of nucleotides exchanged, in comparison to the input DNA sequences (i.e. starting point homologous DNA sequences).

An important advantage of the invention is that mosaic DNA sequences with multiple replacement points or replacements, not related to the opening site, is created, which is not discovered in Pompon's method.

An other important advantage of the present invention is that when using a mixture of fragments and opened vectors (in the screening set up) it gives the possibility of many different clones to recombine pairwise or even triplewise (as can be seen in a couple of examples below).

The in vivo recombination method of the invention simple to perform and results in a high level of mixing of homologous genes or variants. A large number of variants or homologous genes can be mixed in one transformation. The mixing of improved variants or wild type genes followed by screening increases the number of further improved variants manyfold compared to doing only random mutagenesis.

Recombination of multiple overlapping fragments is possible with a high efficiency increasing the mixing of variants or homologous genes using the in vivo recombination method. An overlap as small as 10 bp is sufficient for recombination which may be utilized for very easy domain shuffling of even distantly related genes.

The invention relates to a method for preparing polypeptide variants by shuffling different nucleotide sequences of homologous DNA sequences by in vivo recombination comprising the steps of
a) forming at least one circular plasmid comprising a DNA sequence encoding a polypeptide,
b) opening said circular plasmid(s) within the DNA sequence(s) encoding the polypeptide(s),
c) preparing at least one DNA fragment comprising a DNA sequence homologous to at least a part of the polypeptide coding region on at least one of the circular plasmid(s), d) introducing at least one of said opened plasmid(s), together with at least one of said homologous DNA fragment(s) covering full-length DNA sequences encoding said polypeptide(s) or parts thereof, into a recombination host cell,
e) cultivating said recombination host cell, and
f) screening for positive polypeptide variants.

According to the invention more than one cycle of step a) to f) may be performed.

The opening of the plasmid(s) in step b) can be directed toward any site within the polypeptide coding region of the plasmid. The plamid(s) may be opened by any suitable methods known in the art. The opened ends of the plasmid may be filled-in with nucleotides as described in Pompon et al. (1989), supra). It is preferred not to fill in the opened ends as it might create a frameshift.

It is preferred to open the plasmid(s) around the middle of the polypeptide coding DNA sequence(s), as this is believed to result in a more effective recombination between DNA fragment(s) and opened plasmid(s).

In an embodiment of the invention the DNA fragment(s) is(are) prepared under conditions resulting in a low, medium or high random mutagenesis frequency.

To obtain low mutagenesis frequency the DNA sequence(s) (comprising the DNA fragment(s)) may be prepared by a standard PCR amplification method (U.S. Pat. No. 4,683,202 or Saiki et al., (1988), Science 239, 487-491).

A medium or high mutagenesis frequency may be obtained by performing the PCR amplification under conditions which increase the misincorporation of nucleotides, for instance as described by Deshler, (1992), GATA 9(4), 103-106; Leung et al., (1989), Technique, Vol. 1, No. 1, 11-15.

It is also contemplated according to the invention to combine the PCR amplification (i.e. according to this embodiment also DNA fragment mutation) with a mutagenesis step using a suitable physical or chemical mutagenizing agent, e.g., one which induces transitions, transversions, inversions, scrambling, deletions, and/or insertions.

In the context of the present invention the term “positive polypeptide variants” means resulting polypeptide variants possessing functional properties which has been improved in comparison to the polypeptides producible from the corresponding input DNA sequences. Examples, of such improved properties can be as different as e.g. biological activity, enzyme washing performance, antibiotic resistance etc.

Consequently, which screening method to be used for identifying positive variants depend on the desired improved property of the polypeptide variant in question.

If, for instance, the polypeptide in question is an enzyme and the desired improved functional property is the wash performance, the screening in step f) may conveniently be performed by use of a filter assay based on the following principle:

The recombination host cell is incubated on a suitable medium and under suitable conditions for the enzyme to be secreted, the medium being provided with a double filter comprising a first protein-binding filter and on top of that a second filter exhibiting a low protein binding capability. The recombination host cell is located on the second filter. Subsequent to the incubation, the first filter comprising the enzyme secreted from the recombination host cell is separated from the second filter comprising said cells. The first filter is subjected to screening for the desired enzymatic activity and the corresponding microbial colonies present on the second filter are identified.

The filter used for binding the enzymatic activity may be any protein binding filter e.g. nylon or nitrocellulose. The topfilter carrying the colonies of the expression organism may be any filter that has no or low affinity for binding proteins e.g. cellulose acetate or DuraporeÔ. The filter may be pre-treated with any of the conditions to be used for screening or may be treated during the detection of enzymatic activity.

The enzymatic activity may be detected by a dye, fluorescence, precipitation, pH indicator, IR-absorbance or any other known technique for detection of enzymatic activity.

The detecting compound may be immobilized by any immobilizing agent e.g. agarose, agar, gelatine, polyacrylamide, starch, filter paper, cloth; or any combination of immobilizing agents.

If the improved functional property of the polypeptide is not sufficiently good after one cycle of shuffling, the polypeptide may be subjected to another cycle.

In an embodiment of the invention at least one shuffling cycle is a backcrossing cycle with the initially used DNA fragment, which may be the wild-type DNA fragment. This eliminates non-essential mutations. Non-essential mutations may also be eliminated by using wild-type DNA fragments as the initially used input DNA material.

It is to be understood that the method of the invention is suitable for all types of polypeptide, including enzymes such as proteases, amylases, lipases, cutinases, amylases, cellulases, peroxidases and oxidases.

Also contemplated according to the invention is polypeptides having biological activity such as insulin, ACTH, glucagon, somatostatin, somatotropin, thymosin, parathyroid hormone, pigmentary hormones, somatomedin, erythropoietin, luteinizing hormone, chorionic gonadotropin, hypothalamic releasing factors, antidiuretic hormones, thyroid stimulating hormone, relaxin, interferon, thrombopoietin (TPO) and prolactin.

Especially contemplated according to the present invention is initially to use input DNA sequences being either wild-type, variant or modified DNA sequences, such as a DNA sequences coding for wild-type, variant or modified enzymes, respectively, in particular enzymes exhibiting lipolytic activity.

In an embodiment of the invention the lipolytic activity is a lipase activity derived from the filamentous fungi of the Humicola sp., in particular Humicola lanuginosa, especially Humicola lanuginosa.

In a specific embodiment of the invention the initially used input DNA fragment to be shuffled with a homologous polypeptide is the wild-type DNA sequence encoding the Humicola lanuginosa lipase derived from Humicola lanuginosa DSM 4109 described in EP 305 216 (Novo Nordisk A/S).

Also specifically encompassed by the scope of the invention is input DNA sequences selected from the group of vectors (a) to (f) and/or DNA fragments (g) to (aa) coding for Humicola lanuginosa lipase variants from the list below in the Material and Method section.

Throughout the present application the name Humicola lanuginosa has been used to identify one preferred parent enzyme, i.e. the one mentioned immediately above. However, in recent years H. lanuginosa has also been termed Thermomyces lanuginosus (a species introduced the first time by Tsiklinsky in 1989) since the fungus show morphological and physiological similarity to Thermomyces lanuginosus. Accordingly, it will be understood that whenever reference is made to H. lanuginosa this term could be replaced by Thermomyces lanuginosus. The DNA encoding part of the 18S ribosomal gene from Thermomyces lanuginosus (or H. lanuginosa) have been sequenced. The resulting 18S sequence was compared to other 18S sequences in the GenBank database and a phylogenetic analysis using parsimony (PAUP, Version 3.1.1, Smithsonian Institution, 1993) have also been made. This clearly assigns Thermomyces lanuginosus to the class of Plectomycetes, probably to the order of Eurotiales. According to the Entrez Browser at the NCBI (National Center for Biotechnology Information), this relates Thermomyces lanuginosus to families like Eremascaceae, Monoascaceae, Pseudoeurotiaceae and Trichocomaceae, the latter containing genera like Emericella, Aspergillus, Penicillium, Eupenicillium, Paecilomyces, Talaromyces, Thermoascus and Sclerocleista.

Consequently, such genes encoding lipolytic enzymes of filamentous fungi of the genera Emericella, Aspergillus, Penicillium, Eupenicillium, Paecilomyces, Talaromyces, Thermoascus and Scierocleista are also specifically contemplated according to the present invention.

Other examples of relevant filamentous fungi genes encoding lipolytic enzymes include strains of the Absidia sp. e.g. the strains listed in WO 96/13578 (from Novo Nordisk A/S) which are hereby incorporated by reference. Absidia sp. strains listed in WO 96/13578 include Absidia blakesleeana, Absidia corymbifera and Absidia reflexa.

Strains of Rhizopus sp., in particular Rh. niveus and Rh. oryzea are also contemplated according to the invention.

The lipolytic gene may also be derived from a bacteria, such as a strain of the Pseudomonas sp., in particular Ps. fragi, Ps. stutzeri, Ps. cepacia and Ps. fluorescens (WO 89/04361), or Ps. plantarii or Ps. gladioli (U.S. Pat. No. 4,950,417) or Ps. alcaligenes and Ps. pseudoalcaligenes (EP 218 272, EP 331 376, or WO 94/25578 (disclosing variants of the Ps. pseudoalcaligenes lipolytic enzyme), the Pseudomonas sp. variants disclosed in EP 407 225, or a Pseudomonas sp. lipolytic enzyme, such as the Ps. mendocina (also termed Ps. putida) lipolytic enzyme described in WO 88/09367 and U.S. Pat. No. 5,389,536 or variants thereof as described in U.S. Pat. No. 5,352,594, or Ps. auroginosa or Ps. glumae, or Ps. syringae, or Ps. wisconsinensis (WO 96/12012 from Solvay) or a strain of Bacillus sp., e.g. the B. subtilis described by Dartois et al., (1993) Biochemica et Biophysica acta 1131, 253-260, or B. stearothermophilus (JP 64/7744992) or B. pumilus (WO 91/16422) or a strain of Streptomyces sp., e.g. S. scabies, or a strain of Chromobacterium sp. e.g., C. viscosum.

In connection with the Pseudomonas sp. lipases it has been found that lipases from the following organisms have a high degree of homology, such as at least 60% homology, at least 80% homology or at least 90% homology, and thus are contemplated to belong to the same family of lipases: Ps. ATCC21808, Pseudomonas sp. lipase commercially available as Liposam©, Ps. aeruginosa EF2, Ps. aeruginosa PAC1R, Ps. aeruginosa PAO1, Ps. aeruginosa TE 3285, Ps. sp. 109, Ps. pseudoalcaligenes M1, Ps. glumae, Ps. cepacia DSM 3959, Ps. cepacia M-12-33, Ps. sp. KWI-56, Ps. putida IFO 3458, Ps. putida IFO 12049 (Gilbert, E. J., (1993), Pseudomonas lipases: Biochemical properties and molecular cloning. Enzyme Microb. Technol., 15, 634-645). The species Pseudomonas cepacia has recently been reclassified as Burkholderia cepacia, but is termed Ps. cepacia in the present application.

Also genes encoding lipolytic enzymes from yeasts are relevant, ans include lipolytic genes from Candida sp., in particular Candida rugosa, or Geotrichum sp., in particular Geotrichum candidum.

Specific examples of microorganisms comprising genes encoding lipolytic enzymes used for commercially available products and which may serve as donor of genes to be shuffled according to the invention include Humicola lanuginosa, used in Lipolase®, Lipolase® Ultra, Ps. mendocina used in Lumafast®, Ps. alcaligenes used in Lipomax®, Fusarium solani, Bacillus sp. (U.S. Pat. No. 5,427,936, EP 528828), Ps. mendocina, used in Liposam®.

It is to be emphasized that genes encoding lipolytic enzyme to be shuffled according to the invention may be any of the above mentioned genes of lipolytic enzymes and any variant, modification, or truncation thereof. Examples of such genes which are specifically contemplated include the genes encoding the enzymes described in WO 92/05249, WO 94/01541, WO 94/14951, WO 94/25577, WO 95/22615 and a protein engineered lipase variants as described in EP 407 225; a protein engineered Ps. mendocina lipase as described in U.S. Pat. No. 5,352,594; a cutinase variant as described in WO 94/14964; a variant of an Aspergillus lipolytic enzyme as described in EP patent 167,309; and Pseudomonas sp. lipase described in WO 95/06720.

A request to the DNA sequences, encoding the polypeptide(s), to be shuffled, is that they are at least 60%, preferably at least 70%, better more than 80%, especially more than 90%, and even better up to almost 100% homologous. DNA sequences being less homologous will have less inclination to interact and recombine.

Also the Pseudomonas sp. lipase gene shown in SEQ ID NO. 14 are specifically contemplated according to the invention.

It is also contemplated according to the invention to shuffle parent (homologous) wildt type organisms of different genera.

Further, the DNA fragment(s) to be shuffled may preferably have a length of from about 20 bp to 8 kb, preferably about 40 bp to 6 kb, more preferred about 80 bp to 4 kb, especially about 100 bp to 2 kb, to be able to interact optimally with the opened plasmid.

The method of the invention is very efficient for preparing polypeptide variants in comparison to prior art method comprising transforming linear DNA fragments/sequences.

The inventor found that the transformation frequency of a mixture of opened plasmid and a DNA fragment were significantly higher than when transforming a plasmid cut at the same site alone. The transformation frequency of the opened plasmid and DNA fragment were as high as for uncut plasmid.

Without being limited to any theory it is believed that the opening of the plasmid(s) restrict(s) the replication of (opened) plasmid(s) when not interacting with at least one DNA fragment. In accordance with this an increased number of recombined DNA sequences were found after only one shuffling cycle.

As described in Example 150% of the resulting transformants contained recombined DNA sequences of both input DNA sequences. As high as 20% of the total number of recombined DNA sequences were “random” mixtures (i.e. having more than one region of nucleotides exchanged).

The input DNA sequences may be any DNA sequences including wild-type DNA sequences, DNA sequences encoding variants or mutants, or modifications thereof, such as extended or elongated DNA sequences, and may also be the outcome of DNA sequences having been subjected to one or more cycles of shuffling (i.e. output DNA sequences) according to the method of the invention or any other method (e.g. any of the methods described in the prior art section).

When using the method of the invention the output DNA sequences (i.e. shuffled DNA sequences), have had a number of nucleotide(s) exchanged. This results in replacement of at least one amino acid within the polypeptide variant, if comparing it with the parent polypeptide. It is to be understood that also silent mutations is contemplated (i.e. nucleotide exchange which does not result in changes in the amino acid sequence).

However, the method of the present invention will in most cases lead to the replacement of a considerable number of amino acid and may in certain cases even alter the structure of one or more polypeptide domains (ie. a folded unit of polypeptide structure).

According to the present invention more than two DNA sequences are shuffled at the same time. Actually any number of different DNA fragments and homologous polypeptides comprised in suitable plasmids may be shuffles at the same time. This is advantageous as a vast number of quite different variants can be made rapidly without an abundance of iterative procedures.

The inventor have tested the nucleotide shuffling method of the invention using significantly more than two homologous DNA sequences. As described in Example 2 it was surprisingly found that the method of the invention advantageously can be used for recombining more than two DNA sequences.

One cycle of shuffling according to the method of the invention may result in the exchange of from 1 to 1000 nucleotides into the opened plasmid DNA sequence encoding the polypeptide in question. The exchanged nucleotide sequence(s) may be continuous or may be present as a number of sub-sequences within the full-length sequence(s).

To support the present invention the inventor made a number of additional experiments on different aspect on the method of the invention. The experiments are described below and illustrated in the Example 3 to 6 below.

A number of vectors and fragments comprising an inactivated synthetic Humicola lanuginosa lipase genes were constructed by introducing frameshift/stop codon mutations in the lipase gene at various positions. These were used for monitoring the in vivo recombination of different combinations of opened vector(s) and DNA fragments. The number of active lipase colonies were scored as described in Example 3. The number of colonies determines the efficiency of the opened vector(s) and fragment(s) recombination.

One frameshift mutation in said Humicola lanuginosa lipase gene in the opened vector and another in the fragment on the opposite side of the opening site gave 3 to 32% of active lipase colonies depending on the location and combination. It was concluded that the closer that the mutation is at the ends of the vector the higher mixing.

One frameshift mutation in the opened vector and two in the fragment on each side of the opening site gave 4 to 42% of active colonies depending on the location and combination. Some of these active colonies can be considered to be mosaics, not only related to the opening site.

Two frameshift mutations in the opened vector on each side of the opening site and one in the fragment gave 0.5 to 3.1% of active colonies depending on the location and combination. Most of these active colonies are mosaics of the “parent” DNA.

Two frameshift mutations in the opened vector on each side of the opening site and a wild type fragment gave 7.7 to 10.7% of active colonies depending on the location.

It was also found that the amount of vectors relative to fragments and the size of the fragments are also influencing the result.

Using of the S. cerevisiae rad52 mutants as the recombination host cell showed that the rad52 mutant transformed very well with wild type plasmid(s) and expressed the Humicola lanuginosa lipase gene, but gave no transformants at all with the opened vectors and fragments.

The RAD52 function is required for “classical recombination” (but not for unequal sister-strand mitotic recombination) showing that the recombination of opened vector and fragment could involve a classical recombination mechanism.

Classical recombination is the recombination mechanism involved in the recombination between genes located on nonsister chromatids of homologous chromosomes as defined in for example Petes T D, Malone R E and Symington L S (1991) “Recombination in Yeast”, page 407-522, in The Molecular and Cellular Biology of the Yeast Saccharomyces, Volume 1 (eds. Broach J R, Pringle J R and Jones E W), Cold Spring Harbor Laboratory Press, New York.

Multiple Partially Overlapping Fragements

The inventor also tested recombination of multiple partial overlapping fragments using the method of the invention.

The recombination of 2 and 3 partial overlapping fragments into a gapped (i.e. that the opening result in cutting out of a little part of the gene) vector were tested and gave a high recovery of recombined Humicola lanuginosa lipase gene. The recovery of active lipase gene from different combinations of inactivated Humicola lanuginosa genes was tested for the recombination of 2 partial overlapping fragments. The tendency was a higher mixing in the overlapping region between the 2 fragments in the gapped region than in the vector and fragment overlap.

When recombining many fragments from the same region, the multiple overlapping fragment technique will increase the mixing by itself, but it is also important to have a relative high random mixing in overlapping regions in order to mix closely located variants/differences.

An overlap as small as 10 bp between two fragments were found to be sufficient to obtain a very efficient recombination. Therefore, overlapping in the range from 5 to 5000 bp, preferably from 10 bp to 500 bp, especially 10 bp to 100 bp is suitable according to the method of the invention.

According to this embodiment of the present invention 2 or more overlapping fragments, preferable 2 to 6 overlapping fragments, especially 2 to 4 overlapping fragments may advantageously be used as input fragments in a shuffling cycle.

Besides increasing the mixing of genes, this is a very useful method for domain shuffling by creating small overlaps between DNA fragments from different domains and screen for the best combination.

For instance, in the case of three DNA fragments the overlapping regions may be as follows:
the first end of the first fragment overlaps the first end of the opened plasmid,
the first end of the second fragment overlaps the second end of the first fragment, and the second end of the second fragment overlaps the first end of the third fragment,
the first end of the third fragment overlaps (as stated above) the second end of the second fragment, and the second end of the third fragment overlaps the second end of the opened plasmid.

It is to be understood that when using two or more DNA fragments as starting material it is preferred to have continuos overlaps between the ends of the plasmid and the DNA fragments.

Even though it is preferred to shuffle homologous DNA sequences in the form of DNA fragment(s) and opened plasmid(s), it is also contemplated according to the invention to shuffle two or more opened plasmids comprising homologous DNA sequences encoding polypeptides. However, in such case it is compulsory to open the plasmids at different sites.

In an further embodiment of the invention two or more opened plasmids and one or more homologous DNA fragments are used as the starting material to be shuffled. The ratio between the opened plasmid(s) and homologous DNA fragment(s) preferably lie in the range from 20:1 to 1:50, preferable from 2:1 to 1:10 (mol vector:mol fragments) with the specific concentrations being from 1 pM to 10 M of the DNA.

The opened plasmids may advantagously be gapped in such a way that the overlap between the fragments is deleted in the vector in order to select for the recombination).

Preparing the DNA Fragment

The DNA fragment to be shuffled with the homologous polypeptide comprised in an opened plasmid may be prepared by any suitable method. For instance, the DNA fragment may be prepared by PCR amplification (polymerase chain reaction), as described above, of a plasmid or vector comprising the gene of the polypeptide, using specific primers, for instance as described in U.S. Pat. No. 4,683,202 or Saiki et al., (1988), Science 239, 487-491. The DNA fragment may also be cut out from a vector or plasmid comprising the desired DNA sequence by digestion with restriction enzymes, followed by isolation using e.g. electrophoresis.

The DNA fragment encoding the homologous polypeptide in question may alternatively be prepared synthetically by established standard methods, e.g. the phosphoamidite method described by Beaucage and Caruthers, (1981), Tetrahedron Letters 22, 1859-1869, or the method described by Matthes et al., (1984), EMBO Journal 3, 801-805. According to the phosphoamidite method, oligonucleotides are synthesized, e.g. in an automatic DNA synthesizer, purified, annealed, ligated and cloned in suitable vectors.

Furthermore, the DNA fragment may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire DNA sequence, in accordance with standard techniques.

The Plasmid

The plasmid comprising the DNA sequence encoding the polypeptide in question may be prepared by ligating said DNA sequence into a suitable vector or plasmid, or by any other suitable method.

Said vector may be any vector which may conveniently be subjected to recombinant DNA procedures. The choice of vector will often depend on the recombination host cell into which it is to be introduced.

Thus, the vector may be an autonomously replicating vector, i.e. a vector which exists as an extrachromosomal entity, the replication of which is independent of chromosomal replication, e.g. a plasmid. Alternatively, the vector may be one which, when introduced into the recombination host cell, is integrated into the host cell genome and replicated together with the chromosome(s) into which it has been integrated.

To facilitate the screening process it is preferred that the vector is an expression vector in which the DNA sequence encoding the polypeptide in question is operably linked to additional segments required for transcription of the DNA. In general, the expression vector is derived from a plasmid, a cosmid or a bacteriophage, or may contain elements of any or all of these.

The term, “operably linked” indicates that the segments are arranged so that they function in concert for their intended purposes, e.g. transcription initiates in a promoter and proceeds through the DNA sequence coding for the polypeptide in question.

The promoter may be any DNA sequence which shows transcriptional activity in the recombination host cell of choice and may be derived from genes encoding proteins, such as enzymes, either homologous or heterologous to the host cell.

Examples of suitable promoters for use in yeast host cells include promoters from yeast glycolytic genes (Hitzeman et al.,(1980), J. Biol. Chem. 255, 12073-12080; Alber and Kawasaki, (1982), J. Mol. Appl. Gen. 1, 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., (1983), Nature 304, 652-654) promoters.

Examples of suitable promoters for use in filamentous fungus host cells are, for instance, the ADH3 promoter (McKnight et al., (1985), The EMBO J. 4, 2093-2099) or the tpiA promoter. Examples of other useful promoters are those derived from the gene encoding A. oryzae TAKA amylase, Rhizomucor miehei aspartic proteinase, A. niger neutral a-amylase, A. niger acid stable a-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters.

The DNA sequence encoding polypeptide in question invention may also, if necessary, be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 E1b region), transcriptional enhancer sequences (e.g. the SV40 enhancer) and translational enhancer sequences (e.g. the ones encoding adenovirus VA RNAs).

The vector may further comprise a DNA sequence enabling the vector to replicate in the recombination host cell in question.

When the host cell is a yeast cell, suitable sequences enabling the vector to replicate are the yeast plasmid 2m replication genes REP 1-3 and origin of replication.

The plasmid pY1 can be used for production of useful proteins and peptides, using filamentous fungi, such as Aspergillus sp., and yeasts as recombinant host cells (JP06245777-A).

The vector may also comprise a selectable marker, e.g. a gene the product of which complements a defect in the recombination host cell, such as the gene coding for dihydrofolate reductase (DHFR) or the Schizosaccharomyces pombe TPI gene (described by P. R. Russell, (1985), Gene 40, 125-130).

Another example of such suitable selective markers are the ura3 and leu2 genes which complements the corresponding defect genes of e.g. the yeast strain Saccharomyces cerevisiae YNG318.

The vector may also comprise a selectable marker which confers resistance to a drug, e.g. ampicillin, kanamycin, tetracyclin, chloramphenicol, neomycin, hygromycin or methotrexate. For filamentous fungi, selectable markers include amdS, pvrG, argB, niaD, sC, trpC, pyr4, and DHFR.

To direct the polypeptide in question into the secretory pathway of the recombination host cell, a secretory signal sequence (also known as a leader sequence, prepro sequence or pre sequence) may be provided in the recombinant vector. The secretory signal sequence is joined to the DNA sequence encoding the lipolytic enzyme in the correct reading frame. Secretory signal sequences are commonly positioned 5′ to the DNA sequence encoding the polypeptide. The secretory signal sequence may be the signal normally associated with the polypeptide in question or may be from a gene encoding another secreted protein.

The signal peptide may be naturally occurring signal peptide, or a functional part thereof, or it may be a synthetic peptide. For secretion from yeast cells, suitable signal peptides have been found to be the a-factor signal peptide (cf. U.S. Pat. No. 4,870,008), the signal peptide of mouse salivary amylase (cf. 0. Hagenbuchle et al., (1981), Nature 289, 643-646), a modified carboxypeptidase signal peptide (cf. L. A. Valls et al., (1987), Cell 48, 887-897), the Humicola lanuginosa lipase signal peptide, the yeast BAR1 signal peptide (cf. WO 87/02670), or the yeast aspartic protease 3 (YAP3) signal peptide (cf. M. Egel-Mitani et al., (1990), Yeast 6, 127-137).

For efficient secretion in yeast, a sequence encoding a leader peptide may also be inserted downstream of the signal sequence and upstream of the DNA sequence encoding the polypeptide in question. The function of the leader peptide is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The leader peptide may be the yeast a-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease, a Humicola lanuginosa lipase. The signal peptide is preferably derived from a gene encoding A. oryzae TAKA amylase, A. niger neutral α-amylase, A. niger acid-stable amylase, or A. niger glucoamylase.

The Recombination Host Cell

The recombination host cell, into which the mixture of plasmid/fragment DNA sequences are to be introduced, may be any eukaryotic cell, including fungal cells and plant cells, capable of recombining the homologous DNA sequences in question.

According to prior art prokaryotic microorganisms, such as bacteria including Bacillus and E. coli; eukaryotic organisms, such as filamentous fungi, including Aspergillus and yeasts such as Saccharomyces cerevisiae; and tissue culture cells from avian or mammalian origins have been suggested for in vivo recombination. All of said organisms can be used as recombination host cell, but in general prokaryotic cells are not sufficiently effective (i.e. does not result in a sufficient number of variants) to be suitable for recombination methods for industrial use.

Consequently, preferred recombination host cells according to the present invention are fungal cells, such as yeast cells or filamentous fungi.

Examples of suitable yeast cells include cells of Saccharomyces sp., in particular strains of Saccharomyces cerevisiae or Saccharomyces kluyveri or Schizosaccharomyces sp., Methods for transforming yeast cells with heterologous DNA and producing heterologous polypeptides therefrom are described, e.g. in U.S. Pat. No. 4,599,311, U.S. Pat. No. 4,931,373, U.S. Pat. No. 4,870,008, 5,037,743, and U.S. Pat. No. 4,845,075, all of which are hereby incorporated by reference. Transformed cells may be selected by, e.g., a phenotype determined by a selectable marker, commonly drug resistance or the ability to grow in the absence of a particular nutrient, eg. leucine. A preferred vector for use in yeast is the POT1 vector disclosed in U.S. Pat. No. 4,931,373. The DNA sequence encoding the polypeptide may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al.,(1986), J. Gen. Microbiol. 132, 3459-3465; U.S. Pat. No. 4,882,279).

Examples of other fungal cells are cells of filamentous fungi, e.g. Aspergillus sp., Neurospora sp., Fusarium sp. or Trichoderma sp., in particular strains of A. oryzae, A. nidulans or A. niger. The use of Aspergillus sp. for the expression of proteins is described in, e.g., EP 272 277, EP 230 023. The transformation of F. oxysporum may, for instance, be carried out as described by Malardier et al., (1989), Gene 78, 147-156.

In a preferred embodiment of the invention the recombination host cell is a cell of the genus Saccharomyces, in particular S. cerevisiae.

Methods and Materials

DNA Sequence:

Humicola lanuginosa DSM 4109 derived lipase encoding DNA sequence.

Humicola lanuginosa lipase variants:

Variants Used for Preparing Vectors to be Opened With NruI in Example 2:

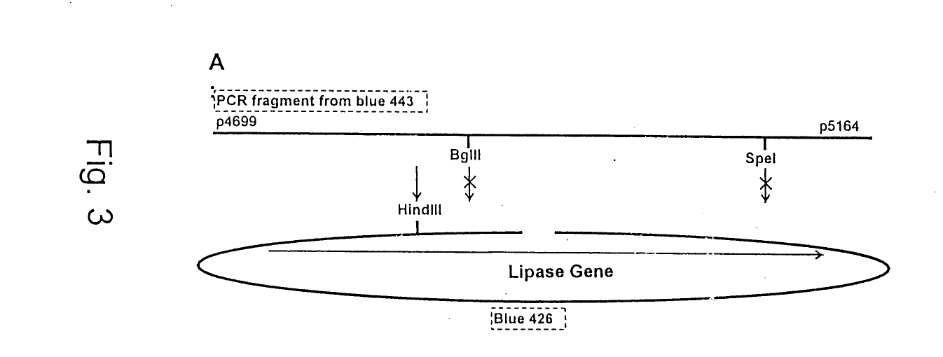

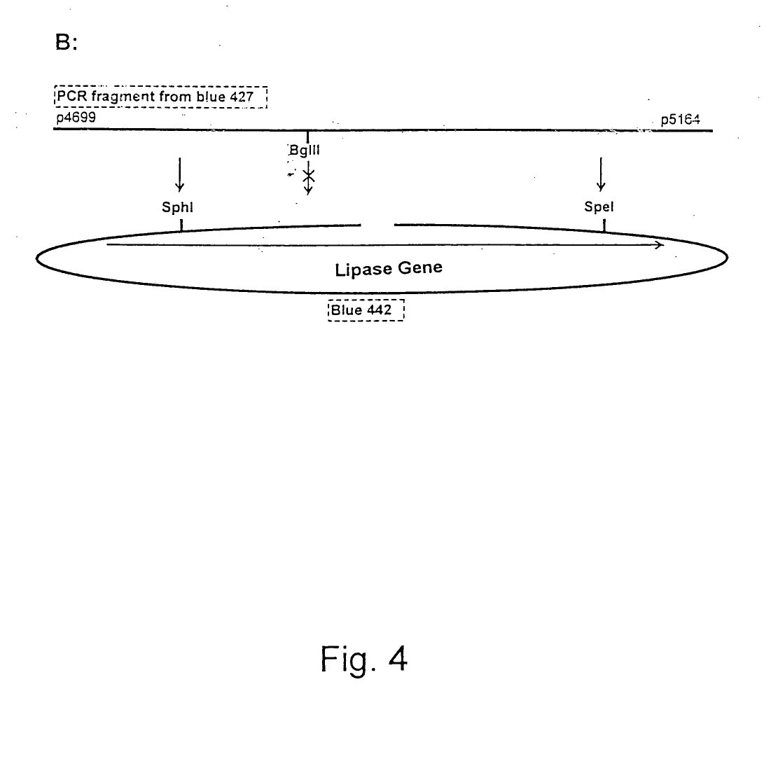

(a) E56R,D57L,I90F,D96L,E99K
(b) E56R,D57L,V60M,D62N,S83T,D96P,D102E
(c) D57G,N94K,D96L,L97M
(d) E87K,G91A,D96R,I100V,E129K,K237M,I252L,P256T,G263A,L264Q
(e) E56R,D57G,S58F,D62C,T64R,E87G,G91A,F95L,D96P,K98I,(K237M)
(f) E210K
   Variants Used for Preparing DNA Fragments by Standard PCR Amplification in Example 2:
(g) S83T,N94K,D96N
(h) E87K,D96V
(i) N94K,D96A
(j) E87K,G91A,D96A
(k) D167G,E210V
(l) S83T,G91A,Q249R
(m) E87K,G91A
(n) S83T,E87K,G91A,N94K,D96N,D111N.
(o) N73D,E87K,G91A,N941,D96G.
(p) L67P,I76V,S83T,E87N,I90N,G91A,D96A,K98R.
(q) S83T,E87K,G91A,N92H,N94K,D96M
(s) S85P,E87K,G91A,D96L,L97V.
(t) E87K,I90N,G91A,N94S,D96N,I100T.
(u) 134V,S54P,F80L,S85T,D96G,R108W,G109V,D111G,S116P,L124S, V132M,V140Q,V141A,F142S,H145R,N162T,I166V,F181P,F183S,R205G, A243T,D254G,F262L.
(v) E56R,D57L,I90F,D96L,E99K
(x) E56R,D57L,V60M,D62N,S83T,D96P,D102E
(y) D57G,N94K,D96L,L97M
(z) E87K,G91A,D96R,I100V,E129K,K237M,I252L,P256T,G263A,L264Q
(aa) E56R,D57G,S58F,D62C,T64R,E87G,G91A,F95L,D96P,K98I
   Strains:
   Expression System Host:
   Saccharomyces cerevisiae YNG318: MATa Dpep4[cir⁺] ura3-52, leu2-D2, his 4-539 Saccharomyces cerevisiae Rad52: Strain M1533=MATa rad52 ura3, obtained from Torsten Nilsson Tillgren, Institute of Genetics, University of Copenhagen.
   Plasmids:
pJSO26 (see FIG. 3)
pJSO37 (see FIG. 4)
pYES 2.0 (Invitrogen)
   Transformation Selective Marker
ura3
leu2
   Media
SC-ura⁻: 90 ml 10× Basal salt, 22.5 ml 20% casamino acids, 9 ml 1% tryptophan, H₂O ad 806 ml, autoclaved, 3.6 ml 5% threonine and 90 ml 20% glucose or 20% galactose added.
LB-medium: 10 g Bacto-tryptone, 5 g Bacto yeast extract, 10 g NaCl in 1 liter water.
Brilliant Green (BG) (Merck, art. No. 1.01310)
BG-reagent: 4 mg/ml Brilliant Green (BG) dissolved in water
Substrate 1:
   10 ml olive oil (Sigma CAT NO. 0-1500)
   20 ml 2% polyvinyl alcohol (PVA)
   The Substrate is homogenised for 15-20 minutes.
      Methods:
      Construction of Yeast Expression Vector

The expression plasmids pJSO26 and pJSO37, are derived from pYES 2.0. The inducible GAL1-promoter of pYES 2.0 was replaced with the constitutively expressed TPI (triose phosphate isomerase)-promoter from Saccharomyces cerevisiae (Albert and Karwasaki, (1982), J. Mol. Appl Genet., 1, 419-434), and the ura3 promoter has been deleted. A restriction map of pJSO26 and pJSO37 is shown in FIG. 3 and FIG. 4, respectively.

Preparation of the Wild-Type DNA Fragment

A lipase wild-type DNA fragment can be prepared either by PCR amplification (resulting in low, medium or high mutagenesis), of the pJSO26 plasmid or by cutting the DNA fragment out by digesting with a suitable restriction enzyme.

Fermentation of Humicola lanuginosa Lipase Variants in Yeast

10 ml of SC-ura³¹ medium is inoculated with a S. cerevisiae colony and grown at 30° C. for 2 days. The 10 ml is used for inoculating 300 ml SC-ura⁻ medium which is grown at 30° C. for 3 days. The 300 ml is used for inoculation 5 l of the following G-substrate:

| | |
|---|---|
| 400 g | Amicase |
| 6.7 g | yeast extract (Difco) |
| 12.5 g | L-Leucin (Fluka) |
| 6.7 g | (NH₄)₂SO₄ |
| 10 g | MgSO₄.7H₂O |
| 17 g | K₂SO₄ |
| 10 ml | Trace compounds |
| 5 ml | Vitamin solution |
| 6.7 ml | H₃PO₄ |
| 25 ml | 20% Pluronic (antifoam) |

In a Total Volume of 5000 ml:

The yeast cells are fermented for 5 days at 30° C. They are given a start dosage of 100 ml 70% glucose and added 400 ml 70% glucose/day. A pH=5.0 is kept by addition of a 10% NH₃ solution. Agitation is 300 rpm for the first 22 hours followed by 900 rpm for the rest of the fermentation. Air is given with 1 l air/l/min for the first 22 hours followed by 1.5 l air/l/min for the rest of the fermentation.

Trace Compounds:

| | |
|---|---|
| 6.8 g | ZnCl₂ |
| 54.0 g | FeCl₂.6H₂O |
| 19.1 g | MnCl₂.4H₂O |
| 2.2 g | CuSO₄.5H₂O |
| 2.58 g | CoCl₂ |
| 0.62 g | H₃BO₃ |
| 0.024 g | (NH₄)₆Mo₇O₂₄.4H₂O |
| 0.2 g | KI |
| 100 ml | HCl (concentrated) |

In a total volume of 1 l.

Vitamin Solution:

| | |
|---|---|
| 250 mg | Biotin |
| 3 g | Thiamin |
| 10 g | D-Calciumpanthetonat |
| 100 g | Myo-Inositol |
| 50 g | Cholinchloride |
| 1.6 g | Pyridoxin |
| 1.2 g | Niacinamide |
| 0.4 g | Folicacid |
| 0.4 g | Riboflavin |

In a total volume of 1 l.

Transformation of Yeast

Saccharomyces cerevisiae is transformed by standard methods (cf. Sambrooks et al., (1989), Molecular Cloning: A Laboratory Manual, 2nd Ed., Cold Spring Harbor)

Determination of Yeast Transformation Frequency

The transformation frequency is determined by cultivating the transformants on SC-ura⁻plates for 3 days and counting the number of colonies appearing. The number of transformants per mg opened plasmid is the transformation frequency.

Screening for Positive Variants With Improved Wash Performance

The following filter assay can be used for screening positive variants with improved wash performance.

Low Calcium Filter Assay

1) Provide SC Ura⁻ replica plates (useful for selecting strains carrying the expression vector) with a first protein binding filter (Nylon membrane) and a second low protein binding filter (Cellulose acetate) on the top.
2) Spread yeast cells containing a parent lipase gene or a mutated lipase gene on the double filter and incubate for 2 or 3 days at 30° C.
3) Keep the colonies on the top filter by transferring the top-filter to a new plate.
4) Remove the protein binding filter to an empty petri dish.
5) Pour an agarose solution comprising an olive oil emulsion (2% PVA:olive oil=3:1), Brilliant green (indicator,0.004%), 100 mM tris buffer pH9 and EGTA (final concentration 5 mM) on the bottom filter so as to identify colonies expressing lipase activity in the form of blue-green spots.
6) Identify colonies found in step 5) having a reduced dependency for calcium as compared to the parent lipase.
   DNA sequencing was performed by using applied Biosystems ABI DNA sequence model 373A according to the protocol in the ABI Dye Terminator Cycle Sequencing kit.
   Assessing the Effiency of Recombination

The number of colonies determines the efficiency of the opened vector and fragment recombination. The percentage of colonies with active lipase activity gives an estimate of the mixing of the active and inactive genes—theoretically it can be calculated for one frameshift that the closer to 50% the better mixing if equal likelihood of wild type and frameshift, 25% for 2 frameshifts and 12.5% for 3 frameshifts.

Frameshift Mutation

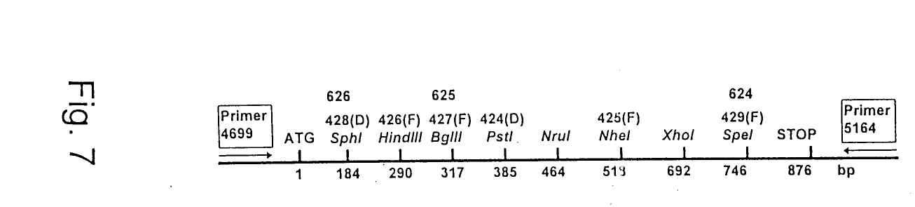

The frameshift mutation were created either by filling in a restriction site (in case of 5′ overhang) or deleting the “sticky ends” (in case of 3′ overhang) by T4 DNA polymerase with or without dNTP (deoxynucleotides=equal amounts of dATP, dTTP, dCTP and dGTP). Methods for filling in of restriction sites (referred to as “F” on FIG. 7) and deleting the sticky ends (referred to as “(D))” on FIG. 7) are well known in the art.

Method for Assessing Colonies With Lipase Activity

The number of colonies and positives (Le. with lipase activity) are calculated as the average of 3 plates.

The cultivation condition and screening condition used is the following:
1) Provide SC Ura-plates with a protein binding filter (Nylon filter) onto the plate.
2) Spread yeast cells containing a parent lipase gene or a mutated lipase gene on the filter and incubate for 3 or 4 days at 30° C.
3) Remove the protein binding filter with the colonies to a petri dish containing: An agarose solution comprising an olive oil emulsion (2% PVA:Olive oil=2:1), Brilliant green (indicator,0.004%), 100 mM tris buffer pH 9.
5) Identify colonies expressing lipase activity in the form of blue-green spots.

### EXAMPLES

### Example 1

Testing In Vivo Recombination of Two Homologous Genes

The Saccharomyces cerevisiae expression plasmid pJSO26 was constructed as described above in the “Material and Methods”-section.

A synthetic Humicola lanuginosa lipase gene (in pJSO37) containing 12 additional restriction sites (see FIG. 4) was cut with NruI, PstI, and NruI and PstI, respectively, to open the gene approximately in the middle of the DNA sequence encoding the lipase.

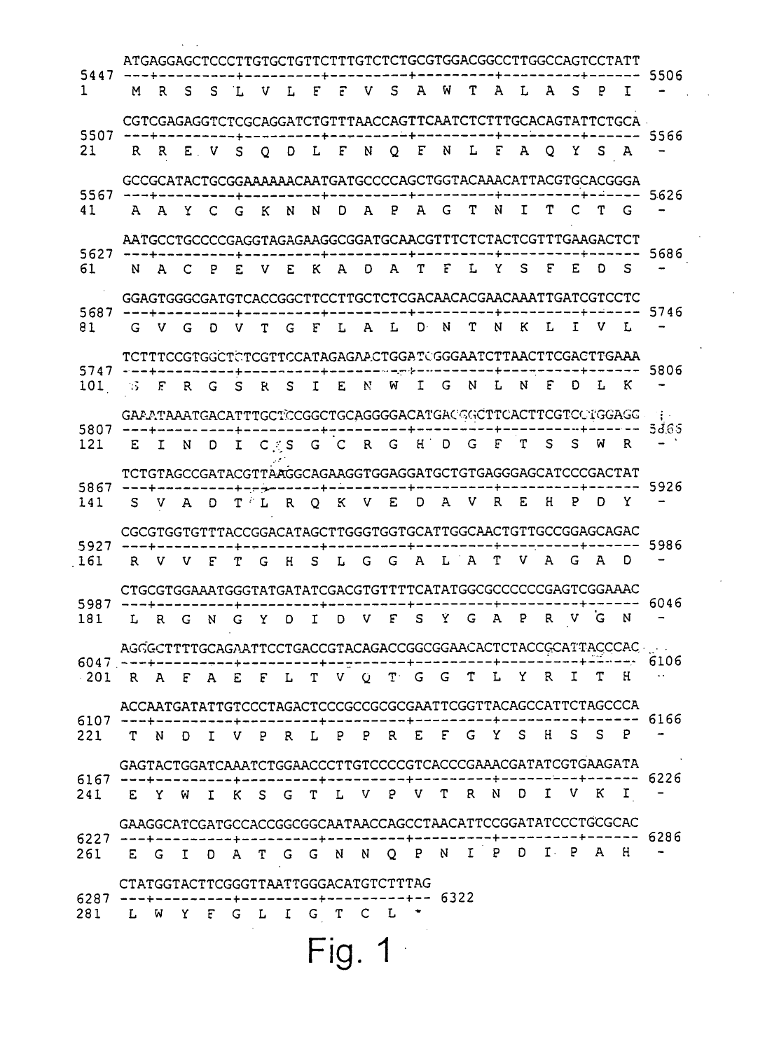

The opened plasmid (pJSO37) was transformed into Saccharomyces cerevisiae YNG318 together with an about 0.9 kb wild-type Humicola lanuginosa lipase DNA fragment (see FIG. 1) prepared from pJSO26 by PCR amplification.

Further, the opened plasmid was also transformed into the yeast recombination host cell alone (ie. without the 0.9 kb synthetic lipase DNA fragment).

The transformed yeast cells were grown as described in the “Materials and Method”-section above, and the transformation frequency was determined as described above.

It was found that the transformation frequency of the opened plasmid alone was very low (10 transformants per mg opened plasmid), in comparison to the transformation frequency of said plasmid/fragment (50,000 transformants per mg opened plasmid).

The plasmid/fragment was PCR amplified resulting in 20 transformants containing fragments covering the lipase gene region of the recombined plasmid/fragments. The recombination mixture of the 20 transformants were analyzed by restriction site digestion using standard methods. The result is displayed in Table 1.

TABLE 1
NruI (not tested)

| PCR | | | | | | | | |
|---|---|---|---|---|---|---|---|---|
| fragment | SphI | HindIII | PstI | BstXI | NhI | BstEII | KpnI | XhoI |
| P1 | wt | wt | wt | wt | wt | wt | wt | wt |
| P2 | sg | sg | sg | wt | wt | wt | wt | wt |
| P3 | sg | sg | sg | sg | nd | sg | sg | nd |
| P4 | nd | sg | sg | wt | nd | wt | nd | nd |
| P5 | wt | wt | nd | wt | wt | wt | wt | wt |
| P6 | sg | sg | sg | sg | sg | sg | sg | nd |
| N1 | wt | wt | wt | wt | sg | wt | wt | wt |
| N2 | wt | wt | wt | wt | wt | wt | wt | wt |
| N3 | wt | wt | wt | wt | wt | wt | wt | wt |
| N4 | sg | sg | sg | wt | wt | wt | wt | wt |
| N5 | sg | sg | sg | wt | wt | wt | wt | wt |
| N6 | wt | wt | wt | sg | sg | sg | sg | sg |
| P/N1 | sg | sg | sg | wt | wt | wt | wt | wt |
| P/N2 | sg | sg | sg | sg | sg | sg | sg | nd |
| P/N3 | sg | sg | sg | wt | nd | sg | sg | sg |
| P/N4 | sg | sg | sg | sg | sg | sg | sg | nd |
| P/N5 | sg | sg | sg | sg | sg | sg | sg | nd |
| P/N6 | sg | sg | sg | wt | nd | sg | sg | sg |
| P/N7 | nd | wt | wt | wt | nd | wt | nd | wt |
| P/N8 | sg | sg | sg | wt | wt | wt | sg | nd |

P: plasmid opened with PstI
N: Plasmid opened with NRuI
P/N: plasmid opened with PstI and NRuI (resulting in the removal of a 75 bp fragment)
wt: wild-type gene restriction enzyme pattern
sg: synthetic gene restriction enzyme pattern
nd: not determined

As can be seen from Table 1 10 transformants (equivalent to 50%) contained recombined DNA sequences. 4 of these 10 DNA sequences (equivalent to 20%) contained either a region of the wild-type gene recombined into the synthetic gene or a region of the synthetic gene recombined into the wild-type fragment.

### Example 2

In Vivo Recombination of Humicola lanuginosa Lipase Variants

The DNA sequences of 20 variants of the Humicola lanuginosa lipase were in vivo recombined in the same mixture.

Six vectors were prepared from the lipase variants (a) to (f) (see the list above) by ligation into the yeast expression vector pJSO37. All vectors were cut open with Nrul.

DNA fragment of all 20 homologous DNA sequences (g) to (aa) (see the list above) were prepared by PCR amplification using standard methods.

The 20 DNA fragments and the 6 opened vectors were mixed and transformed into the yeast Saccharomyces cerevisiae YNG318 by standard methods. The recombination host cell was cultivated as described above and screened as described above. About 20 transformants were isolated and tested for improved wash performance using the filter assay method described in the “Material and Methods”-section.

Two positive transformants (named A and B) were identified using the filter assay.

In comparison to the wild-type amino acid sequence the two recombined positive transformants had the following mutations.

| A: | D57G, N94K, D96L, P256T |
|---|---|
| | ------- ------- ------ ==== |

A is a recombination of two variants.
---- originates from the vector (d)
==== originates from the DNA fragment prepared from variant (y)

| B: | D57G, G59V, N94K, D96L, L97M, S116P, S170P, N249R |
|---|---|
| | ---- ???? ------ ------ ------- <<< ????? ==== |

B is a recombination of vector (c), DNA fragments (n) and (u).
---- originates from the vector (c)
<<<< originates from the DNA fragment prepared from variant (u)
==== originates from the DNA fragment prepared from variant (n)
???? Amino acid mutation which is not a result of recombination.

As can be seen the resulting positive variants have been formed by recombination two or more variants. The amino acid mutations marked “?????” are not a result of in vivo recombination, as none of the shuffled lipase variants (see the list above) comprise any of said mutations. Consequently, these mutations are a result of random mutagenesis arisen during preparation of the DNA fragments by standard PCR amplification.

### Example 3

Recombination With One Frameshift Mutantions

Synthetic Humicola lanuginosa lipase gene (in vector JSO37) was made inactive at various positions by deleting (positions 184/385) or filling-in (position 290/317/518/746) restriction enzyme sites or by site-directed introduction of a stop codon. All inactive synthetic lipase genes of 900 bp can be deduced from FIG. 7).

A number of different 900 bp DNA fragments were made from the above vectors using primer 4699 and primer 5164 using standard PCR technique. Smaller PCR fragments were made using primer 8487 and primer 4548 (260 bp), primer 2843 and primer 4548 (488 bp).

0.5 ml (app. 0.1 mg) of vectors Blue 425, Blue 426, Blue 428 and Blue 429, opened with Pst I (i.e. position 385), vectors Blue 424 and Blue 425 opened with NruI (i.e. position 464) were together with 3 ml (app. 0.5 mg) of fragments 424, 425, 426, 428, 429 in varios combination transformed into 100 ml Sacchromyces cerevisiae YNG318 competent cells as displayed in Table 1A.

The number of colonies and positives (i.e. with lipase activity) were calculated as the average of 3 plates as described in the Material and Methods section.

The result of the test is shown in Table 1A

TABLE 1A

| | Number of | % of colonies with |
|---|---|---|
| vector + Fragment | colonies | active lipase activity |
| 1. Blue 428 + 429 [symbol] | 774 | 16% |
| 2. Blue 429 + 428# | 645 | 3% |
| 3. Blue 426 + 425# | 276 | 25% |
| 4. Blue 425 + 426 | 528 | 18% |
| 5. Blue 425/NruI + 426 | 539 | 28% |
| 6. Blue 425 + 424 | 139 | 7% |
| 7. Blue 424/NruI + 425 [symbol] | 74 | 32% |
| 8. Blue 428 + 425 | 81 | 12% |
| 9. Blue 428 + wt fragment | 317 | 37% |

Pairwise recombinations of one frameshift mutation on the vector and another on the fragment on the opposite side of the opening site. [symbol]
determined by 9 plates;
#determined by 6 plates.

The first 2 rows of Table 1A displays vectors and fragments with a frameshift on each side of the PstI site. The “mirror image” experiment in row 2 compared to row 1 gives a reproducible lower number of active colonies. The same is true for row 3 and 4 even though it is not as pronounced. Moving the opening site closer to the frameshift in the vector increases the number of actives as seen in row 5. This can explain the reason for the difference in the “mirror image” experiments. In both cases the higher number of positives has the opening site closer to the frameshift in the vector.

It can therefore be concluded that the closer the mutation is to the end of the vector the higher chance of mixing. This is probably arising from the well known fact that free DNA ends have a high recombinogenic potential. Therefore it is desirable to have as many free DNA ends as possible to increase the mixing of the genes. This is for example obtained in the later example with recombination of multiple overlapping fragments.

Row 6 has a rather low number of actives probably due to the location of the frameshift on the fragment exactly at the PstI opening site of the vector.

Row 7 has the frameshift of the vector close to the opening site and again it gives a high number of actives.

Recombination With One Stop Codon Mutantions

In order to test if there are any difference in the recombination efficiency of stop codon mutations compared to frameshift mutations the following experiments were made.

The same way as described above 0.5 ml (app. 0.1 mg) vectors Blue 624, Blue 625 and Blue 626 (see Table 1B) opened with PstI comprising stop codons at specified positions (positions 184, 317 and 746, respectively) (perpared by site-directed mutagenesis) were together with 3 ml (app. 0.5 mg) of fragments 624, 625 and 626 transformed into 100 ml Sacchromyces cerevisiae YNG318 competent cells in varios combination as displayed in Table 1B.

TABLE 1B

| | | % of colonies with |
|---|---|---|
| Vector + Fragment | Number of colonies | lipase activity |
| 1. Blue 626 + 624 | ND | 40% |
| 2. Blue 624 + 626 | ND | 12% |
| 3. Blue 625 + 624 | ND | 75% |
| 4. Blue 624 + 625 | ND | 10% |

Pairwise recombinations of one stop codon mutation on the vector and another on the fragment on the opposite side of the opening site.
ND = not determined but a high number.

Row 1 and 2 (in Table 1B) have the mutations located at the same place as row 1 and 2 in Table 1A. As can be seen the number of colonies with lipase activity is clearly higher for the stop codon mutations compared to the frameshift mutations, but the same relative difference between the “mirror image” experiments.

This might indicate that the stop codon mutations, which is closer to the “application” of the method, gives a better mixing than frameshift mutations. Row 3 and 4 confirms that the closer the mutation is to the end of the vector the higher chance of mixing.

Recombination With One or Two Frameshift Mutation in the Vector and One or Two Frameshift Mutations in the Fragment

Using the same approach as described above the influence of one or two frameshift mutations in the vector and one or two frameshift mutations in the fragment were tested using vectors Blue 425, 426 and 428 (one mutation) and vectors Blue 442, Blue 443 (two mutations) and fragments 442 and 443 (two frameshift mutations) and fragments 424, 425, 426, 427, 428 (one mutation) and wild-type (no mutation).

The vectors Blue 442 and 443 are double frameshift mutations: Blue 442=428+429 and blue 443=427+429 (see FIG. 7).

Recombination was performed by transforming 0.5 ml vector (app. 0.1 mg) opened with PstI and 3 ml PCR-fragment (app. 0.5 mg) into 100 ml Sacchromyces cerevisiae YNG318 competent cells.

The result of the test is shown in Table 2A and Table 2B

TABLE 2A

| | | % of colonies with |
|---|---|---|
| Vector + Fragment | Number of colonies | active Lipolase |
| 1. Blue 425 + 442 | 142 | 15% |
| 2. Blue 425 + 443 | 144 | 14% |
| 3. Blue 426 + 442 | 42 | 42% |
| 4. Blue 426 + 443# | 77 | 20% |
| 5. Blue 428 + 443 | 115 | 3.8% |

One frameshift mutation on the vector and two on the fragment on each side of the opening site.
#determined by 6 plates.

TABLE 2B

| | | % of colonies with active |
|---|---|---|
| Vector + Fragment | Number of colonies | Lipolase |
| Blue 442 + 424 | 137 | 0.5% |
| Blue 442 + 426 | 118 | 1.1% |
| Blue 442 + 427# | 125 | 1.3% |
| Blue 443 + 425 | 540 | 2.5% |
| Blue 443 + 426 | 196 | 1.5% |
| Blue 443 + 428 | 469 | 3.1% |
| Blue 442 + wt fragment | 135 | 7.7% |
| Blue 443 + wt fragment | 488 | 10.7% |

Two frameshift mutations on the vector on each side of the opening site and one on the fragment.
#determined by 6 plates.

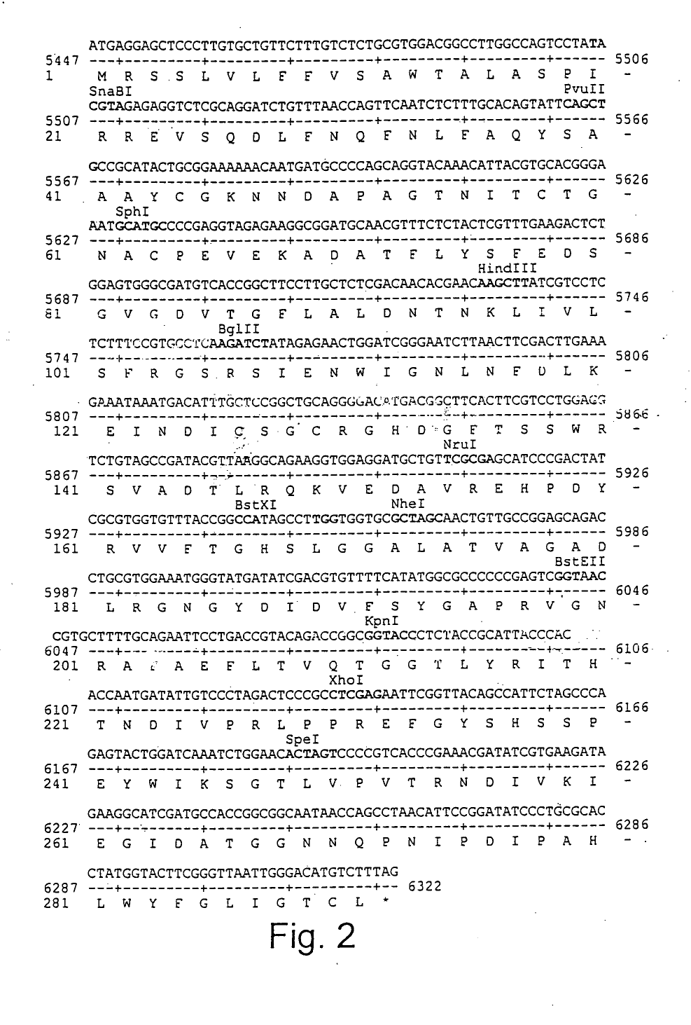

Table 2A shows a rather high number of colonies with lipase activity even with a total of 3 frameshifts (but only one frameshift on the vector) except for the last row where the frameshift on the vector is located far from the opening site. Lane 4 has fewer actives than lane 3 probably due to that the frameshift on the vector is located further away from the opening site than the frameshift on the fragment making the active genes mosaics that are not related to the opening site (see FIG. 2A). In Table 2B a very low number of actives are observed when there are 2 frameshifts located on the vector. Most of these active colonies are mosaics of the “parent” DNA meaning that the mixing is not related to the opening site (see FIG. 2B).

Recombination With Two Different Vectors or Fragments

The result of recombination with two different vectors or fragnments the test is shown in Table 3

TABLE 3

| | Number of | % of colonies with |
|---|---|---|
| Vector + Fragment | colonies | active Lipolase |
| Blue 428/pstI + Blue | 13 | 15% |
| 429/pst# | | |
| Blue428/pst + Blue | 273 | 4.2% |
| 429/PstI + 442 | | |
| Blue 442/pstI + 428 + 429 | 228 | 0.8% |
| Blue 443/pstI + 427 + 428 | 229 | 1.6% |

Recombinations with 2 different vectors or fragments.
#Determined by 1 plate.

A low number of colonies are seen for the control experiment in row 1 of table 3 as expected. The fragment added in the middle row has two frameshifts each corresponding to the frameshift on each vector. Via a tripartite recombination 4.2% actives are created. With two fragments with each one frameshifts and a vector with the same two frameshifts very few actives are found.

Recombination With Vectors Opened at Different Sites

Opening the vector in one side instead of approximately in the middle still gives good recombination as shown in Table 4. Two vectors opened at different sites can also recombine to some extent (compare with the vector controls in table 13).

TABLE 4

| | Number of | % of colonies with |
|---|---|---|
| Vector + Fragment | colonies | active Lipolase |
| Blue 428/xho + 429 | 160 | 11% |
| Blue 428/xho + Blue 429/pst# | 35 | 6.3% |

Opening of the vector in one side instead of in the middle.
#determined by 6 plates.

Recombination at Different Concentrations of Vector and Fragment

The relative concentration of vector to fragment do influence the percentage of positive colonies as can be seen in Table 5.

TABLE 5

| | Number of | % of colonies with |
|---|---|---|
| Vector + Fragment | colonies | lipase activity |
| 0.5 μl Blue 426 + 3 μl 442 | 42 | 42% |
| 1.5 μl Blue 426 + 3 μl 442 | 21 | 51% |
| 1.5 μl Blue 426 + 9 μl 442 | 34 | 26% |
| 1.5 μl Blue 426 + 3 μl 427 | 230 | 2.8% |
| 1 μl Blue 442 + 1 μl 425 | 224 | 1.16% |
| 1 μl Blue 442 + 2 μl 425 | 429 | 0.9% |
| 1 μl Blue 442 + 4 μl 425 | 434 | 1.6% |
| 1 μl Blue 442 + 8 μl 425 | 481 | 1.6% |
| 1 μl Blue 442 + 16 μl 425 | 497 | 2.0% |

Varying the concentration of the vector or fragment.

Recombination With Fragments of Different Size

The size of the fragment also influences the recombination result as seen in Table 6.

TABLE 6

| | Number of | % of colonies with |
|---|---|---|
| Vector + Fragment | colonies | active Lipolase |
| Blue 424 + 425 (260 bp) | 73 | 34% |
| Blue 424 + 425 (489 bp) | 130 | 45% |
| Blue 424 + 424 (480 bp) | 133 | 0.3% |
| Blue 424 + 428 (480 bp) | 130 | 36% |
| Blue 428 + 425 (480 bp) | 150 | 28% |
| Blue 425 + 424 (480 bp) | 69 | 0% |
| Blue 425 + 428 (480 bp) | 63 | 55% |

Recombination with smaller fragments than 900 bp.

Recombination With Unopened Vectors

Transformation with unopened vectors shows a very low degree of recombination (Table 7).

TABLE 7

| | Number of | % of colonies with active |
|---|---|---|
| Plasmid | colonies | Lipolase |
| Blue 428 + Blue 429 | 887 | 0.3% |
| Blue 426 + Blue 425 | 697 | 0.7% |

Recombination of unopened plasmids.

### Example 4

Test of S. cerevisiae Mutants Altered in Recombination

Using the same approach as described in Example 3 recombination of opened and unopened vectors and fragments were tested using a Saccharomyces cerevisiae rad52 mutant as the recombination host cell. The result is displayed in Table 8.

TABLE 8

| | Number of | % of colonies with active |
|---|---|---|
| Vector + Fragment | colonies | Lipolase |
| Blue 428 + 429 | 0 | 0 |
| Blue 442 + 427 | 0 | 0 |
| Blue 424 + 425 | 0 | 0 |
| Blue 426 + 443 | 0 | 0 |
| Plasmid pJSO 37 | 544 | 100% |

Recombination result in rad52 mutant.

The result with rad52 showed that recombination was completely abolished. The RAD52 function is required for classical recombination (but not for unequal sister-strand mitotic recombination) showing that the recombination of opened vector and fragment could involve a classical recombination mechanism.

### Example 6

Recombination of Multiple Partial Ping Fragments

In order to increase the mixing of the mutations by the recombination method of the invention, recombination of two fragments and one gapped vector were attempted.

TABLE 15

| | | % of |
|---|---|---|
| | | colonies |
| | Number of | with lipase |
| Vector + Fragment | colonies | activity |
| 1. pJSO37/HindIII-XhoI + PCR319 + PCR327 | >2000 | 100% |
| 2. pJSO37/HindIII-XhoI + PCR321 + PCR331 | ≈2000 | ≈0.2% |
| 3. pJSO37/HindIII-XhoI + PCR319 + PCR331 | ≈1500 | ≈1% |
| 4. pJSO37/HindIII-XhoI + PCR319 + PCR386 | >5000 | >90% |
| 5. pJSO37/HindIII-XhoI + PCR321 + PCR386 | >5000 | ≈25% |
| 6. Blue 428/HindIII-XhoI + PCR321 + PCR331 | 400 | 0.2% |
| 7. Blue 428/HindIII-XhoI + PCR319 + PCR327 | ≈1500 | >90% |
| 8. Blue 428/HindIII-XhoI + PCR321 + PCR327 | ≈150 | ≈10% |
| 9. Blue 428/HindIII-XhoI + PCR327 + PCR385 | ≈1500 | ≈10% |
| 10. Blue 429/HindIII-XhoI + PCR319 + PCR386 | ≈400 | ≈15% |
| 11. Blue 429/HindIII-XhoI + PCR321 + PCR386 | ≈350 | ≈15% |
| 12. Blue 442/HindIII-XhoI + PCR319 + PCR327 | ≈1500 | ≈10% |
| 13. Blue 428/HindIII-XhoI + | 2 | 0 |
| 14. Blue 429/HindIII-XhoI + | 0 | 0 |
| 15. Blue 442/HindIII-XhoI + | 6 | 0 |
| 16. Blue 428/HindIII-XhoI + PCR331 | 4 | 0 |
| 17. Blue 428/HindIII-XhoI + PCR321 | 2 | 0 |

Recombination result of two fragments and a gapped vector. The last 5 rows are controls.

As can be seen in Table 15, the recovery of the Humicola lanuginosa lipase gene is very efficient. The last 5 rows in Table 15 shows that the opened vector alone or with only one fragment not covering the whole gap (see FIG. 3) gives only very few colonies.

The first row is with wild type fragments gives 100% of active colonies.

The second row is with two fragments each containing a frameshift. The fragment PCR331 fragment has the frameshift located at the BglII site which, in this recombination, is not covered by a wild type fragment (see FIG. 3) and therefore gives about 0% of active lipase. The same is the case for row 3 and 6.

In the row 4, fragment PCR386 containing a frameshift at the SphI site which is overlapped by wild type sequences in the gapped vector. The frameshift was recombined into less than 10% of the genes which is lower than the result for one fragment recombination in the last row of Table 1A above.

In row 5 a rather high mixing is observed between the 2 fragments each containing a frameshift and the wild type gapped vector giving 25% active and 75% inactive lipase colonies. This is probably due to that the fragment PCR321 has the frameshift in the overlap between the 2 fragments and in the gapped region of the vector. If fragment PCR386 contributes to 10% inactives like in row 4, fragment PCR321 gives the remaining 65% inactives—therefore PCR386 gives 35% wt in the overlap.

Row 7 is the “mirror image” of row 4 with the frameshift at the SphI site on the vector (see FIG. 7) and 2 wild type fragments giving an integration of the wild type fragment into more than 90% of the vectors.

Row 8 shows like in row 5 that the frameshift of PCR321 in the overlap and gap region gives a very high number of inactive.

In row 9, fragment PCR385 with a frameshift in the vector overlap, causes a very high number of inactives.

Row 10 gives a rather high number of inactives compared to row 7 and 4. It is not increased in row 11.

Row 12 shows that two frameshifts on the vector gives a lower number of actives compared to one in row 7.

The recombination of 3 partial overlapping fragments into a gapped vector is also very efficient as seen in Table 16. The last row with the vector alone gives very few colonies. As can be seen in FIG. 4 all fragments used are wt. In the first row in table 16, there are rather long overlaps between the vector and fragments, but in the middle row the overlap between PCR353 and 355 is only 10 bp long and it is still very efficiently recombined! This surprising result may be utilized for very easy domain shuffling of even distantly related genes. For example can 3 different domains from 10 different genes be made as PCR fragments, designed to have a 10 to 20 bp overlap by primer design and recombined together and subsequently screened for the best combination (1000 possible combinations).

TABLE 16

| | | % of |
|---|---|---|
| | Number | colonies |
| | of | with active |
| Vector + Fragment | colonies | Lipolase |
| pJSO37/PvuII-SpeI + PCR353 + PCR354 + | >5000 | 100% |
| PCR367 | | |
| pJSO37/PvuII-SpeI + PCR353 + PCR355 + | >5000 | 100% |
| PCR367 | | |
| pJSO37/PvuII-SpeI | 20 | 100% |

Recombination result of 3 fragments and a gapped vector. The last row is a control.

1-26. (Cancelled).

27. A method for generating and identifying a shuffled plasmid variant, comprising:
(a) linearizing at least one circular plasmid, wherein the plasmid comprises a DNA sequence encoding a polypeptide of interest and the linearization is within the DNA sequence encoding the polypeptide of interest;
(b) preparing two or more different partially overlapping DNA fragments comprising DNA sequences encoding variants of the polypeptide of interest or parts thereof; wherein the DNA fragments comprise at least one sequence variation within the encoding DNA sequence;
(c) introducing the at least one linearized plasmid of step (a) with the at least two partially overlapping DNA fragments of step (b) into a host cell, wherein recombination occurs between the at least one linearized plasmid and the at least two partially overlapping DNA fragments to generate a recombinant circular plasmid comprising a recombined DNA sequence encoding the polypeptide of interest;
(d) cultivating the host cell comprising the recombinant circular plasmid, wherein the recombined DNA sequence is expressed, and
(e) screening for a positive recombined polypeptide of interest; and
wherein more than one cycle of steps (a)-(c) is performed.

28. The method of claim 27, wherein two or more linearized plasmids are introduced into the host cell.

29. The method of claim 27, wherein each DNA fragment introduced into the host cell encodes a polypeptide having at least 60% homology to the polypeptide of interest.

30. The method of claim 27, wherein the variants of the polypeptide of interest differ by one amino acid.

31. The method of claim 27, wherein the DNA fragments prepared in step (b) are mutagenized.

32. The method of claim 27, wherein the linearized plasmid(s) and DNA fragment(s) are in a ratio of between from 20:1 to 1:50 (moles vector:mole fragment).

33. The method of claim 27 wherein the linearized plasmid(s) and DNA fragment(s) are in a ratio of between from 2:1 to 1:10 with the specific concentrations of from 1 pM to 10 M of the DNA fragment(s).

34. The method of claim 27, wherein the linearized plasmid(s) is gapped.

35. The method of claim 27, wherein the overlapping regions of the DNA fragments are in the range of from 5 to 5000 bp.

36. The method of claim 35, wherein the overlapping regions are in the range of from 10 to 500 bp.

37. The method of claim 35, wherein the overlapping regions are in the range of from 10 to 100 bp.

38. The method of claim 27, wherein at least one subsequent cycle is performed with the same DNA fragments as used in the first cycle.

39. The method of claim 27, wherein the polypeptide of interest is an enzyme or biologically active protein.

40. The method of claim 39, wherein the enzyme is selected from the group consisting of a protease, lipase, cutinase, cellulase, amylase, peroxidase, oxidase, and phytase.

41. The method of claim 27, wherein the DNA sequence encodes a wild-type or variant lipase derived from a filamentous fungus.

42. The method of claim 41, wherein the filamentous fungus is selected from the group consisting of Humicola, Absidia, Rhizopus, Emericella, Aspergillus, Penicillium, Eupenicillium, Paecilomyces, Talaromyces, Thermoascus, Fusarium, and Sclerocleista.

43. The method of claim 41, wherein the lipase is derived from Humicola lanuginosa.

44. The method of claim 43, wherein the lipase is derived from Humicola lanuginosa DSM 4109.

45. The method of claim 27, wherein the host cell is a eukaryotic cell.

46. The method of claim 45, wherein the eukaryotic cell is a fungal cell selected from the group consisting of Saccharomyces sp., Schizosaccharomyces sp., Kluyveromyces sp., Hansenula sp., Pichia sp., Aspergillus sp., Neurospora sp., Fusarium sp., and Trichoderma sp.

47. The method of claim 45, wherein the eukaryotic cell is a fungal cell selected from the group consisting of Saccharomyces cerevisiae, Saccharomyces kluyveri, Schizosaccharomyces pombe, Kluyveromyces lactis, Hansenula polymorpha, Pichia pastoris, Aspergillus niger, Aspergillus nidulans, Aspergillus oryzae, or Fusarium oxysporum.

48. The method of claim 27, wherein the plasmid DNA sequence encoding the polypeptide is operably linked to a functional promoter sequence.

49. The method of claim 48, wherein the plasmid is an expression plasmid.

50. A method for generating and identifying a shuffled plasmid variant, comprising:
(a) linearizing two or more circular plasmids, wherein the plasmids comprise partially overlapping DNA sequences encoding a polypeptide of interest and the linearization is within the DNA sequence encoding the polypeptide of interest; wherein the circular plasmids comprise at least one sequence variation within the encoding DNA sequence;
(b) preparing at least one DNA fragment comprising a DNA sequence encoding the polypeptide of interest or parts thereof;
(c) introducing the linearized plasmids of step (a) with the at least one DNA fragment of step (b) into a host cell, wherein recombination occurs between the two or more linearized plasmids and the at least one DNA fragment to generate a recombinant circular plasmid comprising a recombined DNA sequence encoding the polypeptide of interest;
(d) cultivating the host cell comprising the recombinant circular plasmid, wherein the recombined DNA sequence is expressed; and
(e) screening for a positive recombined polypeptide of interest; and
wherein more than one cycle of steps (a)-(c) is performed


SEQUENCE LISTING (15 sequences)

SEQ ID NO: 1 - DNA (other nucleic acid), 20 bp; single-stranded, linear
  description: /desc = “Primer 2843”
acaaacattacgtgcacggg

SEQ ID NO: 2 - DNA (other nucleic acid), 18 bp; single-stranded, linear
  description: /desc = “Primer 4699”
cggtacccggggatccac

SEQ ID NO: 3 - DNA (other nucleic acid), 18 bp; single-stranded, linear
  description: /desc = “Primer 5164”
aattacatcatgcggccc

SEQ ID NO: 4 - DNA (other nucleic acid), 21 bp; single-stranded, linear
  description: /desc = “Primer 8487”
catttgctccggctgcaggga

SEQ ID NO: 5 - DNA (other nucleic acid), 60 bp; single-stranded, linear
  description: /desc = “Primer 4548”
gtgttccgccggtctgtacggtcaggaattctgcaaaagccctgtttccgactcgggggg

SEQ ID NO: 6 - DNA (other nucleic acid), 21 bp; single-stranded, linear
  description: /desc = “Primer 5576”
ggtctgtacggtcaggaattc

SEQ ID NO: 7 - DNA (other nucleic acid), 19 bp; single-stranded, linear
  description: /desc = “Primer 5578”
cgtttcgggtgacggggac

SEQ ID NO: 8 - DNA (other nucleic acid), 18 bp; single-stranded, linear
  description: /desc = “Primer 1596”
ggagcaaatgtcatttat

SEQ ID NO: 9 - DNA (other nucleic acid), 64 bp; single-stranded, linear
  description: /desc = “Primer 4545”
gcattggcaactgttgccggagcagacctgcgtggaaatgggtatgatatcgacgtgttttcat

SEQ ID NO: 10 - DNA (other nucleic acid), 876 bp; single-stranded, circular
  CDS 1..876
  description: /desc = “Vector pJSO26”
  original source strain: Humicola lanuginosa
atgaggagctcccttgtgctgttctttgtctctgcgtggacggccttggccagtcctattcgtcgagaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttag

SEQ ID NO: 11 - protein, 291 aa
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 12 - DNA (other nucleic acid), 876 bp; single-stranded, circular
  CDS 1..876
  description: /desc = “Vector pJSO37”
  original source strain: Humicola lanuginosa
atgaggagctcccttgtgctgttctttgtctctgcgtggacggccttggccagtcctatacgtagagaggtctcgcaggatctgtttaaccagttcaatctctttgcacagtattcagctgccgcatactgcggaaaaaacaatgatgccccagcaggtacaaacattacgtgcacgggaaatgcatgccccgaggtagagaaggcggatgcaacgtttctctactcgtttgaagactctggagtgggcgatgtcaccggcttccttgctctcgacaacacgaacaagcttatcgtcctctctttccgtggctcaagatctatagagaactggatcgggaatcttaacttcgacttgaaagaaataaatgacatttgctccggctgcaggggacatgacggcttcacttcgtcctggaggtctgtagccgatacgttaaggcagaaggtggaggatgctgttcgcgagcatcccgactatcgcgtggtgtttaccggccatagccttggtggtgcgctagcaactgttgccggagcagacctgcgtggaaatgggtatgatatcgacgtgttttcatatggcgccccccgagtcggtaaccgtgcttttgcagaattcctgaccgtacagaccggcggtaccctctaccgcattacccacaccaatgatattgtccctagactcccgcctcgagaattcggttacagccattctagcccagagtactggatcaaatctggaacactagtccccgtcacccgaaacgatatcgtgaagatagaaggcatcgatgccaccggcggcaataaccagcctaacattccggatatccctgcgcacctatggtacttcgggttaattgggacatgtctttag

SEQ ID NO: 13 - protein, 291 aa; identical to SEQ ID NO: 11

SEQ ID NO: 14 - DNA (genomic), 864 bp; single-stranded, linear
  mat_peptide 1..864
  CDS 1..864
  original source strain: Pseudomonas sp.
ttcggctcctcgaactacaccaagacccagtacccgatcgtcctgacccacggcatgctcggtttcgacagcctgcttggagtcgactactggtacggcattccctcagccctgcgtaaagacggcgccaccgtctacgtcaccgaagtcagccagctcgacacctccgaagcccgaggtgagcaactgctgacccaagtcgaggaaatcgtggccatcagcggcaagcccaaggtcaacctgttcggccacagccatggcgggcctaccatccgctacgttgccgccgtgcgcccggatctggtcgcctcggtcaccagcattggcgcgccgcacaagggttcggccaccgccgacttcatccgccaggtgccggaaggatcggccagcgaagcgattctggccgggatcgtcaatggtctgggtgcgctgatcaacttcctttccggcagcagttcggacaccccacagaactcgctgggcacgctggagtcactgaactccgaaggcgccgcacggtttaacgcccgcttcccccagggggtaccaaccagcgcctgcggcgagggcgattacgtggtcaatggcgtgcgctattactcctggaggggcaccagcccgctgaccaacgtactcgacccctccgacctgctgctcggcgccacctccctgaccttcggtttcgaggccaacgatggtctggtcggacgctgcagctcccggctgggtatggtgatccgcgacaactaccggatgaaccacctggacgaggtgaaccagaccttcgggctgaccagcatcttcgagaccagcccggtatcggtctatcgccagcaagccaatcgcctgaagaacgccgggctc

SEQ ID NO: 15 - protein, 288 aa
FGSSNYTKTQYPIVLTHGMLGFDSLLGVDYWYGIPSALRKDGATVYVTEVSQLDTSEARGEQLLTQVEEIVAISGKPKVNLFGHSHGGPTIRYVAAVRPDLVASVTSIGAPHKGSATADFIRQVPEGSASEAILAGIVNGLGALINFLSGSSSDTPQNSLGTLESLNSEGAARFNARFPQGVPTSACGEGDYVVNGVRYYSWRGTSPLTNVLDPSDLLLGATSLTFGFEANDGLVGRCSSRLGMVIRDNYRMNHLDEVNQTFGLTSIFETSPVSVYRQQANRLKNAGL